(19) United States
(12) Patent Application Publication
(10) Pub. No.: US 2025/0160629 A1
(43) Pub. Date: May 22, 2025

(54) ACCESSORY ACTUATION DEVICES AND RELATED METHODS

(71) Applicant: Boston Scientific Scimed, Inc., Maple Grove, MN (US)

(72) Inventors: Matthew Robert JAGELSKI, Milford, MA (US); Andrew James WHITNEY, Douglas, MA (US)

(73) Assignee: Boston Scientific Scimed, Inc., Maple Grove, MN (US)

(21) Appl. No.: 18/949,075

(22) Filed: Nov. 15, 2024

Related U.S. Application Data
(60) Provisional application No. 63/601,839, filed on Nov. 22, 2023.

(51) Int. Cl. A61B 1/005; A61B 1/00; A61B 1/012

(52) U.S. Cl. CPC A61B 1/0052; A61B 1/00147; A61B 1/0057; A61B 1/012

(57) ABSTRACT

An actuation device comprising: a device body that comprises a first fixation mechanism configured to affix a proximal end of a first member of a medical device to the device body, and an actuator comprising an operable mechanism and a second fixation mechanism configured to affix a proximal end of a second member of the medical device to the actuator. Movement of the operable mechanism from a first position to a second position may be configured to move the second member proximally relative to the first member. Movement of the operable mechanism from the second position to the first position may be configured to move the second member distally relative to the first member. At least one of the first member or the second member of the medical device may be configured to be removably affixed to the device body or the actuator, respectively.

### CROSS-REFERENCE TO RELATED APPLICATIONS

This application claims the benefit of priority of U.S. Provisional Patent Application No. 63/601,839, filed on Nov. 22, 2023, the entirety of which is incorporated herein by reference.

### TECHNICAL FIELD

Aspects of this disclosure generally relate to medical devices and related methods. In particular, aspects of this disclosure relate to accessory actuation devices and methods of using the same.

### BACKGROUND

Medical devices, such as scopes (e.g., endoscopes, duodenoscopes, etc.), may include a handle and a sheath/shaft insertable into a body lumen of a subject. The sheath may terminate in a distal tip portion, which may include features such as elevators, optical elements (e.g., camera, lighting, etc.), air/water outlets, working channel openings, and/or accessory devices. Actuators in a handle of the scope may control actuatable elements of the shaft and/or of the distal tip of the shaft. The sheath may include a working channel, having a distal opening at the distal tip portion and a proximal opening (e.g., a port) in the handle. Various accessory devices may be utilized via the working channel or via an over-the-scope arrangement. However, conventional designs may present challenges when attempting to simultaneously control the endoscope and employ various accessory devices, requiring multiple operators or cumbersome equipment setups.

### SUMMARY

Examples of this disclosure relate to, among other things, an actuation device comprising: a device body that comprises a first fixation mechanism configured to affix a proximal end of a first member of a medical device to the device body, and an actuator comprising an operable mechanism and a second fixation mechanism configured to affix a proximal end of a second member of the medical device to the actuator. Movement of the operable mechanism from a first position to a second position may be configured to move the second member proximally relative to the first member. Movement of the operable mechanism from the second position to the first position is configured to move the second member distally relative to the first member. At least one of the first member or the second member of the medical device may be configured to be removably affixed to the device body or the actuator, respectively.

Any of the devices or systems disclosed herein may have any of the following features. The first fixation mechanism may comprise a first arm and a second arm. The first member may be configured to extend between the first arm and the second arm. The actuator may be pivotally mounted relative to the device body. The actuator may comprise a lever that can move the actuator around a pivot. A portion of the second fixation mechanism extends approximately perpendicularly to a portion of the lever. The device body may be removably fixable to an endoscope handle. The device may further comprise a biasing member configured to bias the actuator in the first position or the second position. The biasing member may be a torsion spring. The second fixation mechanism may comprise a pair of arms, each arm comprising a curved portion. The actuation device may be configured to removably affix to an endoscope handle and the endoscope handle may comprise a female mating member. The device body may comprise a corresponding male mating member for removably coupling the device body to the endoscope handle. The male mating member may be formed of a flexible material. The medical device may be a first medical device. The actuation device may be integrally formed with a handle of a second medical device. The medical device may be a first medical device. The actuation device may be removably fixable to a handle of a second medical device. The first member may be a sheath and the second member may be a pull wire. The second member may include at least one protrusion. The second fixation mechanism includes at least one hook. The medical device may be a first medical device. The first fixation mechanism may be configured to fix the first member of the first medical device relative to a handle of a second medical device. The second fixation mechanism may be movable with respect to the first fixation mechanism, such that the second member of the first medical device is movable with respect to the first member of the first medical device and the handle of the second medical device.

In another aspect, a medical system may comprise: a first medical device having a first handle; an actuation device comprising: a device body that comprises a first fixation mechanism configured to affix a proximal end of a first member of a second medical device to the device body, and an actuator comprising an operable mechanism and a second fixation mechanism configured to affix a proximal end of a second member of the second medical device to the actuator. Movement of the operable mechanism from a first position to a second position may be configured to move the second member relative to the first member. Movement of the operable mechanism from the second position to the first position may be configured to move the second member relative to the first member. The actuation device may be operable from the handle of the first medical device. At least one of the first member or the second member of the second medical device may be configured to be removably affixed to the device body or the actuator.

Any of the systems or devices disclosed herein may have any of the following features. The system may further comprise a third medical device. A first member of the third medical device may be removably affixable to the first fixation member. A second member of the third medical device is affixable to the second fixation member. The second medical device may be one of a tome, a snare, a balloon, a basket, a knife, a needle, a ligation device, a clip, forceps, graspers, a stapler, a suturing device, a stent, a cautery device, or a biopsy collection device, and the third medical device is another of a tome, a snare, a balloon, a basket, a knife, a needle, a ligation device, a clip, forceps, graspers, a stapler, a suturing device, a stent, a cautery device, or a biopsy collection device, different from the second medical device.

In another aspect, an actuation device may comprise: a device body that comprises a first fixation mechanism configured to removably affix a first member of a medical device to the device body, and a lever fixedly coupled to a second fixation mechanism. The second fixation mechanism may be configured to removably affix a proximal end of a second member of the medical device to the lever. Movement of the lever from a first position to a second position may be configured to move the second member proximally relative to the first member. Movement of the lever from the second position to the first position may be configured to move the second member distally relative to the first member.

Any of the devices or systems disclosed herein may have any of the following features. The first fixation mechanism may include at least one prong. The first member may include at least one protrusion. The second fixation mechanism may include at least one hook. The second member may include at least one protrusion.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 shows a side perspective view of a medical device, according to some embodiments.

FIG. 2 shows the medical device of FIG. 1 including a removable actuation device.

FIG. 3 shows a portion of an accessory device for removably coupling to the removable actuation device of FIG. 2.

FIG. 4 shows additional features of the actuation device of FIG. 2.

FIG. 5 shows an embodiment of a coupling mechanism for removably coupling the actuation device of FIG. 2 to a handle of a medical device such as the handle of the medical device of FIG. 1.

FIGS. 6A and 6B show exemplary fixation mechanisms for use with the devices of FIGS. 2-4.

FIG. 7 shows a method of using a medical device, such as the medical device and embodiments shown in FIGS. 1-6.

FIG. 8 shows a medical device with an actuation assembly integrated into the handle of the actuation device.

### DETAILED DESCRIPTION

During an endoscopic procedure, one or more accessory devices may be passed through a working channel of a medical device (e.g., an endoscope) in order to perform a medical procedure. Additionally or alternatively, over-the-scope accessory devices may be utilized. Conventionally, such procedures may require more than one operator (e.g., one operator to operate the endoscope and another operator to actuate the accessory device). Some medical procedures may benefit from being performed by a single operator.

Although the term endoscope may be used herein, it will be appreciated that the disclosure encompasses actuation mechanisms for use with other devices, including, but not limited to, duodenoscopes, colonoscopes, ureteroscopes, bronchoscopes, laparoscopes, cytoscopes, hysteroscopes, sheaths, catheters, any other suitable delivery device, an accessory device, or another type of medical device including an actuator. Although side-facing devices may be referenced, the embodiments described herein may also be used with front-facing devices (e.g., devices where a viewing element faces longitudinally forward) or with devices that are a combination of side-facing and forward-facing. Although the lever assemblies described below are described as being used to actuate an accessory device, it will be appreciated that the assemblies described herein may be used to control other aspects of medical devices.

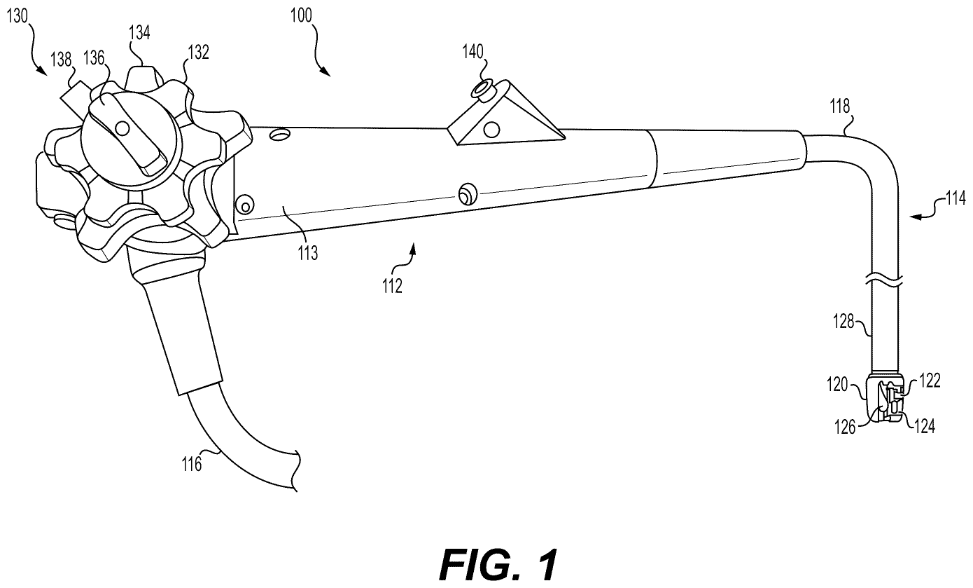

FIG. 1 depicts an exemplary medical device 100 (e.g., an endoscope) which may be a part of a larger medical system (e.g., an endoscope system) and may have a handle 112 and an insertion portion 114. Medical device 100 may also include an umbilicus 116 for purposes of connecting medical device 100 to sources of, for example, air, water, suction, power, etc., as well as to image processing and/or viewing equipment. Insertion portion 114 may include a sheath or shaft 118 and a distal tip 120. Distal tip 120 may include an imaging device 122 (e.g., a camera) and a lighting source 124 (e.g., an LED or an optical fiber). Distal tip 120 may be side-facing. That is, imaging device 122 and lighting source 124 may face radially outward, perpendicularly, approximately perpendicularly, or otherwise transverse to a longitudinal axis of shaft 118 and distal tip 120. Alternatively, distal tip 120 may be forward-facing (imaging device 122 and/or lighting source 124 may face distally).

Distal tip 120 may also include an actuatable element, for example an elevator 126, for changing an orientation of a tool inserted in a working channel of medical device 100. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may have a steerable section 128. Steerable section 128 may include, for example, an articulation joint. Shaft 118 and steerable section 128 may include a variety of structures which are known or may become known in the art. In some examples, one or more steering wires may be coupled to steerable section 128 so that, as the steering wires are tensioned/de-tensioned, steerable section 128 articulates in one or more directions.

Handle 112 may have a housing 113 that encases/houses various elements of handle 112. Handle 112 may have one or more actuators/control mechanisms 130. Control mechanisms 130 may provide control over steerable section 128 or may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include one or more locking mechanisms 136, 138 (e.g., knobs or levers) for preventing steering of steerable section 128 in at least one of an up, down, left, or right direction. Handle 112 may include an elevator control mechanism (not shown). The elevator control mechanism may raise and/or lower elevator 126. For example, the elevator control mechanism may be coupled to a wire, which may be coupled to elevator 126. A port 140 may allow passage of a tool or other type of accessory device through port 140, into a working channel of medical device 100, through shaft 118, to distal tip 120.

In use, an operator may insert at least a portion of shaft 118 into a body lumen of a subject. Distal tip 120 may be navigated to a procedure site in the body lumen. The operator may insert an accessory device, such as an instrument (not shown) into port 140, and pass the accessory device through shaft 118 via a working channel to distal tip 120. The accessory device may exit the working channel at distal tip 120. The user may use the elevator control mechanism to raise elevator 126 and angle the accessory device toward a desired location (e.g., a papilla of the pancreatico-biliary tract). The user may use the accessory device to perform a medical procedure.

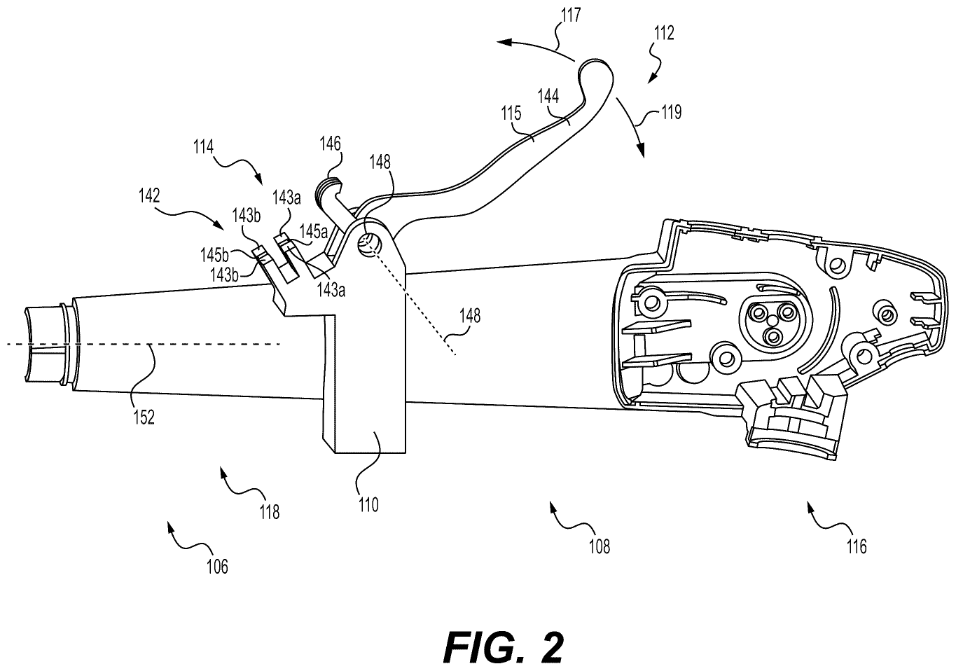

FIG. 2 shows a portion of medical device 100 including the handle 112 and an accessory mounting and actuation device (“actuation device”) 104 for mounting and actuating an accessory device (not shown) to be used with medical device 100. FIG. 2 shows the handle with a portion of the housing 113 removed and without the shaft 118. It will be appreciated that medical device 100 may include the omitted portion of the housing 113 and the shaft 118. The handle 112 has a proximal portion 108 and a distal portion 106. The proximal portion 108 may include features such as the control mechanisms 130. The distal portion 106 may be coupled to the shaft 118.

The actuation device 104 may have a device body 110 that includes a first fixation mechanism 142 that is configured to removably affix a proximal end (or another portion) of a first member (e.g., a tube/sheath/coil pipe) of an accessory device (not shown in FIG. 2) to the device body 110. First fixation mechanism 142 may include any suitable structure for receiving and retaining the first member of the accessory device. For example, first fixation mechanism 142 may include a first, proximal pair of prongs 143a and a second, distal pair of prongs 143b. The first, proximal pair of prongs may be separated from one another by a first, proximal gap 145a. The second, distal pair of prongs may be separated from one another by a second, distal gap 145b. As discussed in further detail below, prongs 143a, 143b may be configured to removably receive and retain the first member.

The actuation device 104 further includes an actuator 115 that includes an operable mechanism 144 (e.g., a lever). Actuator 115 may include a second fixation mechanism 146 that is configured to removably affix to a proximal end/portion of a second member (e.g., an actuation wire or cable) of the accessory device. The second fixation mechanism 146 may include any suitable structure for removably receiving and retaining the second member. For example, the second fixation mechanism 146 may include one or more hooks as shown in FIG. 2. In some examples, the second fixation mechanism 146 may include two hooks that are aligned with one another, with a gap between the hooks. The example of FIG. 2 may include such hooks, although one of the hooks is behind the other hook and not visible in FIG. 2.

The second fixation mechanism 146 may be fixedly coupled to the operable mechanism 144, such that movement of the operable mechanism 144 moves the second fixation mechanism 146. For example, as shown in FIG. 2, the second fixation mechanism 146 may be integrally formed with the operable mechanism 144. As further shown in FIG. 2, the second fixation mechanism 146 may extend at an approximately right angle from a distal end of the operable mechanism 144. However, the arrangement of FIG. 2 is merely exemplary.

Movement of the operable mechanism 144 may actuate one or more features of an accessory device. For example, movement of the operable mechanism 144 may move the second member of the accessory device (e.g., a control member, such as a wire, cable, thread, etc.) relative to the first member of the accessory device (e.g., a sheath, tube, coil pipe, etc. through which the control member extends). Because the first fixation mechanism 142 may fix the first member of the accessory device relative to the handle 112, movement of the operable mechanism 144 (and, thus, the second fixation mechanism 146) may cause the second member of the accessory device to move proximally and/or distally relative to the first member of the accessory device, thereby actuating the accessory device. That is, the second fixation mechanism 146 may be movable with respect to the first fixation mechanism 142 and movement of the second fixation mechanism 146 with respect to the first fixation mechanism 142 may cause the medical device to operator as explained in greater detail herein.

In some examples, the operable mechanism 144 may be pivotable about a pivot axis 148 from a first position to a second position to move the second member of the accessory device proximally relative to the first member of the accessory device as explained in greater detail below. The movement of the operable mechanism 144 from the first position to the second position may be in the direction of arrow 119 in FIG. 2. Movement of the actuation device 104 from the second position to the first position may move the second member of the accessory device distally relative to the first member of the accessory device. The movement of the operable mechanism 144 from the second position to the first position may be in the direction of arrow 117 in FIG. 2. In some embodiments, the pivot axis 148 is perpendicular to a longitudinal axis 152 of the handle 112.

The device body 110 may be removably fixable to the handle 112 and may fix to the handle 112 between the proximal portion 108 and the distal portion 106 of the handle 112. In some examples, the device body 110 may be fixable to the housing 113 proximally of the port 140. The actuation device 104 may be positioned so that an operator may grip the handle 112 in a manner typical to operate the control mechanisms 130, valves of the handle 112, and/or other elements of the handle 112, while reaching the operable mechanism 144 with a finger (e.g., a pinky finger). The device body 110 may be removably affixed to the handle 112 using any means (e.g., a male/female connector as described in greater detail herein below, a strap, a clamp, etc.). The actuation device 104 may be configured so that it can selectively move or be moved between various positions on/along the handle 112.

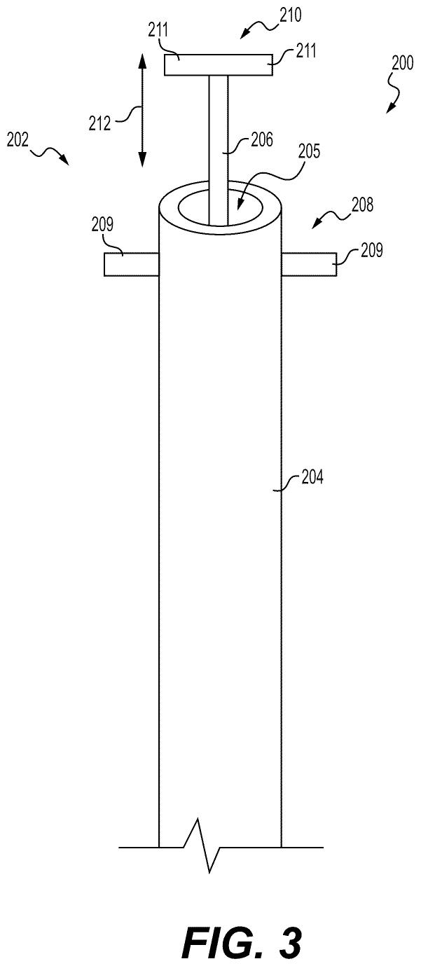

FIG. 3 shows a proximal end portion 202 of an accessory device 200 for removably coupling to the actuation device 104 of FIG. 2. The accessory device 200 may have any feature of any accessory device known in the art. For example, the accessory device may include a tome, a snare, a balloon, a basket, a knife, a needle, a ligation device, a clip, forceps, graspers, a stapler, a suturing device, a stent, a cautery device, a biopsy collection device, or any other accessory device.

The accessory device 200 includes an outer sheath 204 and an inner pull wire 206. The outer sheath 204 is may include a tube, a coil pipe, or any other suitable structure. The outer sheath 204 may define a lumen 205. The inner pull wire 206 is moveably positioned within the lumen 205 of the outer sheath 204. For example, the inner pull wire 206 can move longitudinally in a proximal and/or distal direction with respect to the outer sheath 204, as indicated by arrows 212. Although the term “pull wire” is used herein, it will be appreciated that inner pull wire 206 may also include a cable, a thread, or any other type of control member. The inner pull wire 206 may be coupled to a distal element (not shown), such that proximal and/or distal movement of the inner pull wire 206 relative to the outer sheath 204 may actuate the distal element. It will be readily appreciated by those of skill in the art that pull wire 206 may be used to actuate a wide variety of types of accessory devices, including those listed above.

The outer sheath 204 may include an outer sheath attachment member 208, and the pull wire 206 may include a pull wire attachment member 210. FIG. 3 depicts the outer sheath attachment member 208 as including a pair of protrusions 209 extending outward from the outer sheath 204. For example, the protrusions 209 may extend approximately perpendicularly relative to a longitudinal axis of the outer sheath 204. Alternatively, the protrusions 209 may extend at another transverse (non-parallel) angle with respect to the outer sheath 204 or may extend parallel to the outer sheath 204.

The outer sheath attachment member 208 may be configured to be received by the first fixation mechanism 142 of actuation device 104. For example, the protrusions 209 may be positioned so that they are proximal of the first, proximal pair of prongs 143a or between the first, proximal pair of prongs 143a and the second, distal pair of prongs 143b. The outer sheath 204 may be received within gap 145a and/or 145b. Thus, first fixation mechanism 142 may removably fix the outer sheath 204 relative to the handle 112 of medical device 100. The sheath attachment member 208 and outer sheath 204 may be rotatable when positioned within the first fixation mechanism 142 such that the outer sheath 204 may rotate during insertion of the accessory device 200 within a body lumen of the patient.

The pull wire attachment member 210 may similarly include a pair of protrusions 211 extending outwardly from the pull wire 206. The protrusions 211 may be formed integrally with the pull wire 206 or may be formed from a separate material and coupled to the pull wire 206. As shown in FIG. 3, the protrusions 211 and the pull wire 206 may form an approximately T-shape. In other words, the protrusions 211 may be at a proximal-most end of the pull wire 206. The protrusions 211 may extend approximately perpendicularly relative to a longitudinal axis of the pull wire 206. Alternatively, the protrusions 211 may extend at another transverse (non-parallel) angle with respect to the pull wire 206 or may extend parallel to the pull wire 206.

The pull wire attachment member 210 may be configured to be received by the second fixation mechanism 146 of actuation device 104. For example, the protrusions 211 may be positioned so that they are received proximally of and engage with hooks of the second fixation mechanism 146. The pull wire 206 may extend between the hooks. The curved shape of the hooks of the second fixation mechanism 146 may help to retain the attachment member 210 as operable mechanism 144 pivots. The pull wire 206 may be fixedly coupled to the second fixation mechanism 146 via the pull wire attachment member 210.

With reference to FIGS. 2 and 3, movement of the operable mechanism 144 among two or more positions (e.g., between a first position and a second position) may n move the outer sheath 204 and the inner pull wire 206 with respect to one another. That is, with the pull wire attachment member 210 affixed to the second fixation mechanism 146 and the sheath attachment member 208 affixed to the first fixation mechanism 142, the actuator 115 may cause relative motion between the inner pull wire 206 and the outer sheath 204 via the operable mechanism 144. As discussed above, the second fixation mechanism 146 may pivot along with the operable mechanism 144, thereby moving the second fixation mechanism 146 proximally and distally relative to the handle 112. As discussed above, the first fixation mechanism 142 and the outer sheath 204 may be fixed relative to the handle 112. Thus, movement of the operable mechanism 144 may cause the pull wire 206 to move proximally and/or distally with respect to the outer sheath 204. Although a distal end of the accessory device 200 is not depicted, movement of the pull wire 206 may actuate an element of a distal end of the accessory device, such as for example, forceps, a clip, a snare, a tome device, a balloon, a basket, a knife, a needle, a ligation device, graspers, a stapler, a suturing device, a stent, a cautery device, a biopsy collection device, or any other accessory device, or any other distal end element.

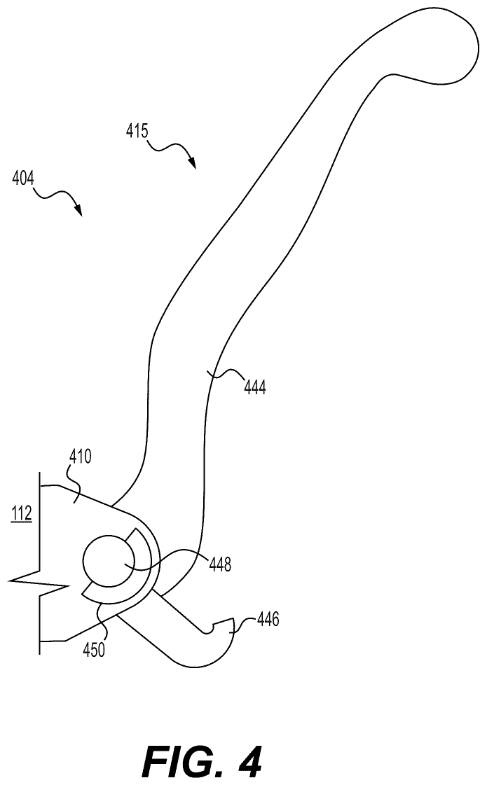

Referring to FIG. 4, a portion of another embodiment of an actuation device 404 is shown. Actuation device 404 may be attached to the handle 112 of medical device 100. Unless otherwise specified herein, the actuation device 404 may include any of the features of the actuation device 104, discussed above. Where feasible, reference numbers relating to the actuation device 404 add 300 to corresponding structures of the actuation device 104 in order to indicate like structures. The actuation device 404 may include a device body 410 and an actuator 415, including an operable mechanism 444. The operable mechanism 444 may be fixedly coupled to a fixation mechanism 446, having any of the features of the second fixation mechanism 146. Although the portion of the actuation device 404 depicted in FIG. 4 does not depict a structure corresponding to the first fixation mechanism 142, it will be appreciated that the actuation device 404 may have such an element on a portion of the actuation device 404 that is not shown in FIG. 4.

The operable mechanism 444 is operable to pivot about a pivot 448. A biasing mechanism 450 biases the operable mechanism 444 toward a biased position. The biasing mechanism 450 can be a torsional spring, a helical spring, a living hinge (e.g., a plastic living hinge), a leaf spring, or another biasing mechanism. A user of medical device 100 can overcome the bias of the biasing mechanism 450 to operate the device by exerting a force on the operable mechanism 444. The biasing mechanism 450 may bias the operable mechanism 444 in any position based on tuning of the biasing mechanism 450, such that the operable mechanism 444 is at rest (in a default, natural position) at one or the other of its limits of rotational movement or somewhere in between. For example, the operable mechanism 444 may be biased distally, such that when a user releases a proximal force on the operable mechanism 444 (a counterclockwise force in FIG. 4), the operable mechanism 444 may return to the distally biased position (rotate clockwise in FIG. 4).

With an accessory device, such as the accessory device 200, attached to the actuation device 404, the pull wire 206 or other control member of the accessory device would then be biased to a natural, resting position based on the bias of the biasing mechanism 450. For example, the pull wire 206 may be biased to a position in which a distal end element of the accessory device 200 has not been actuated. In examples, the pull wire 206 may be biased distally. A user can move features of the accessory device from a biased position toward a forced position, and when the user relieves the force moving the accessory device from the forced position to the biased position, the accessory device will remain in the biased position. In some examples, the biased position may not be a natural resting position of the accessory device.

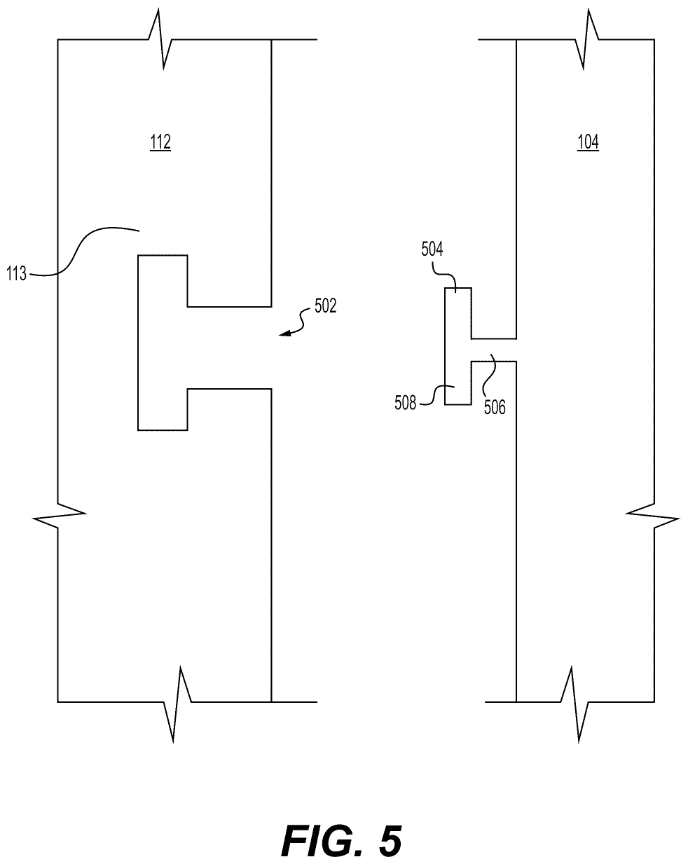

FIG. 5 shows further aspects of medical device 100. The handle 112 may mate with the actuation device 104 via one or more mating features. Although the actuation device 104 is referenced herein, it will be appreciated that the aspects shown in FIG. 5 may be used with the actuation device 404 or any other actuation device described herein. The mating features may include, for example, a male-female mating member. For example, the handle 112 may include a female mating member 502 and the actuation device 104 may include a male mating member 504. As shown in FIG. 5, the male mating member 504 may be a protrusion that extends from the device body 110 of the actuation device 104. The protrusion may have a stem 506 and two protrusions 508 extending outwardly from the stem 506. A shape of the male mating member 504 is merely exemplary, and the male mating member 504 may have any suitable shape. The female mating member 502 may be a recess in the housing 113 of the handle 112. The recess may have a complimentary shape to the male mating member 504. In some examples, the male mating member 504 may be slide by a user into the female mating member 502 in order to mate the actuation device 104 to the handle 112. The male mating member 504 can be made from a flexible material such as, for example, rubber, plastic, or other material.

One or more portions of the male mating member 504 and/or the female mating member 502 may be formed from, for example, a pliable but rigid material (e.g., nylon, Polytetrafluoroethylene (PTFE), polycarbonate, Acrylonitrile Butadiene Styrene (ABS), etc.) enabling the user to removably fix the actuation device 104 to the handle 112. Other mating features are considered for mating the actuation device 104 with the handle 112. For example, the actuation device 104 can removably couple to the handle 112 via one or more hook-and-loop connections, straps, clamps, jaws, slotted connections, keyed connections, twist connections, living plastic springs/clamps, and/or one or more other types of connections.

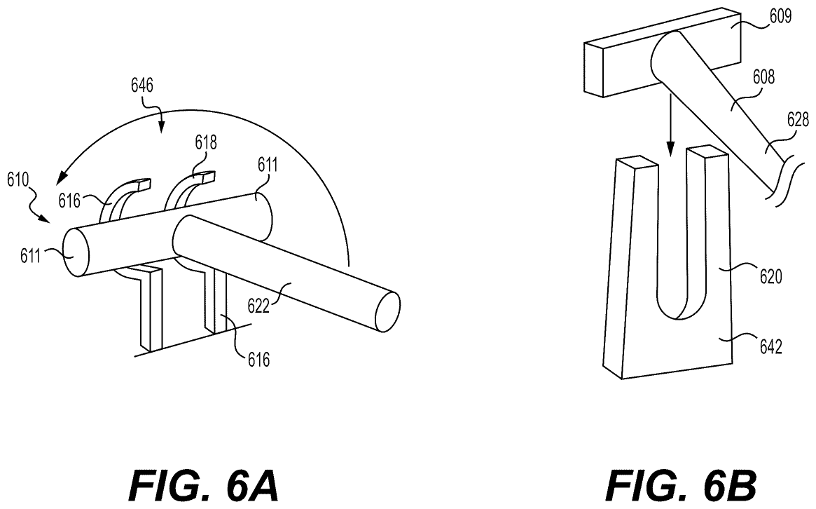

FIGS. 6A and 6B show exemplary mechanisms for connecting an accessory device (for example, an accessory device having the outer sheath 204 and the pull wire 206 of the accessory device 200 of FIG. 3) to a fixation mechanism (for example, the first fixation mechanism 142 and/or the second fixation mechanism 146 of FIG. 2). The mechanisms of FIGS. 6A and 6B may be used in alternative to or in addition to the first fixation mechanism 142, the second fixation mechanism 146, the sheath attachment member 208, and/or the pull wire attachment member 210.

FIG. 6A shows an attachment member 610 (for an accessory device, for example) configured to attach to a fixation mechanism 646. The attachment member 610 may have any of the properties of the pull wire attachment member 210, and the fixation mechanism 646 may have any of the features of the second fixation mechanism 146. The fixation mechanism 646 may include a pair of hooks/arms 616, each having a curved portion 618. The attachment member 610 may have a pair of protrusions 611 (having any of the properties of the protrusions 211) extending from a central shaft 622 (which may be similar to, for example, the pull wire 206 or the outer sheath 204). A user may position the protrusions 611 in the curved portions 618 of the arms 616 and rotate the attachment member 610 in the direction of the arrow such that the central shaft 622 of the attachment member 610 is between the curved portion 618 of the arms 616, thereby securing the attachment member 610 to the fixation mechanism 646. In some examples, the attachment member 610 may be an element of the accessory device 200 (e.g., of the pull wire 206 or the outer sheath 204), and the fixation mechanism 646 may be an element of the actuation device 104 or 404. Alternatively, the attachment member 610 may be an element of the actuation device 104 or 404, and the fixation mechanism 646 may be an element of the accessory device 200.

FIG. 6B depicts an attachment member 608 (for an accessory device, for example) configured to attach to a fixation mechanism 642. The attachment member 608 may have any of the properties of the distal portion 106, and the fixation mechanism 642 may have any of the properties of first fixation mechanism 142. The attachment member 608 may include a t-connector with protrusions (which may form a lateral portion) 609 and a shaft 628. In some examples, the shaft 628 may have any of the properties of the outer sheath 204 or the pull wire 206. The protrusions 609 may be positioned between prongs/arms 620 (which may have any of the properties of the prongs 143a, 143b) of the fixation mechanism 642. In some embodiments, the arms 620 may include one or more features for inhibiting movement of the attachment member 608 into or out of the fixation mechanism 642 (e.g., a bend in a plane of the arms, a snap fit, rough surfaces to enhance a frictional fit, etc.). In some examples, the attachment member 608 may be an element of the accessory device 200 (e.g., of the pull wire 206 or the outer sheath 204), and the fixation mechanism 642 may be an element of the actuation device 104 or 404. Alternatively, the attachment member 608 may be an element of the actuation device 104 or 404, and the fixation mechanism 642 may be an element of the accessory device 200.

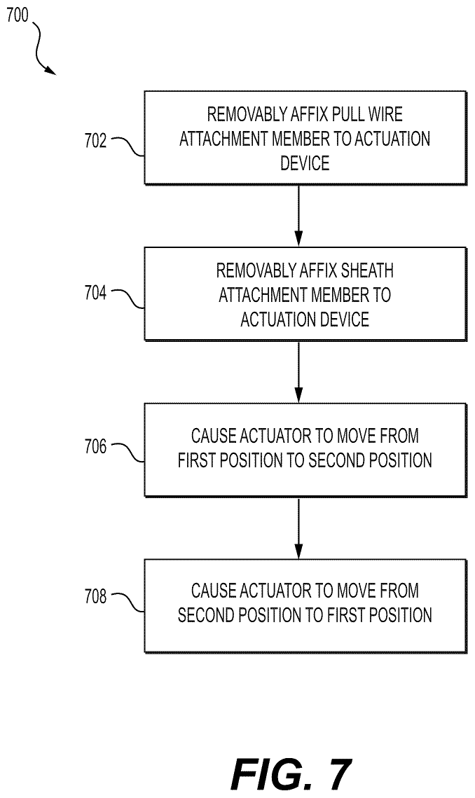

FIG. 7 shows a method 700 of actuating an accessory device using an actuation device. While the method 700 includes the steps shown in FIG. 7, it is to be understood that other methods of actuating an accessory device are within the scope of the method 700 and methods of actuating an accessory device within the scope of this disclosure can include more or fewer steps than the steps shown in FIG. 7.

At step 702, a user may removably affix a pull wire attachment member (including, e.g., the pull wire attachment member 210, or the attachment member 610) of a pull wire (e.g., the pull wire 206 or the central shaft 622) to a fixation mechanism (e.g., the second fixation mechanism 146, the fixation mechanism 446, or the fixation mechanism 646) of a device body (e.g., the device body 110 or the device body 410) of an actuation device (e.g., the actuation device 104 or the actuation device 404). The pull wire may be a pull wire or other control member that is operative to actuate a distal feature of an accessory medical device. The pull wire may extend through an outer sheath (e.g., the outer sheath 204 or the shaft 628). The outer sheath may be inserted into a body lumen of the subject (e.g., patient undergoing treatment with an endoscope) and the pull wire may operatively couple the device to the actuator 115 (e.g., including the operable mechanism 444) for actuating the accessory medical device.

At step 704, a user may affix a sheath attachment member (e.g., the sheath attachment member 208 or the attachment member 608) of an outer sheath (e.g., the outer sheath 204 or the shaft 628) to a first fixation mechanism (e.g., the first fixation mechanism 142 or the fixation mechanism 642) of an actuation device 104. As discussed above, the sheath attachment member may extend outward from an external portion of the outer sheath and may create a friction fit or other removable fit with the first fixation mechanism.

At step 706, a user may actuate an actuator (e.g., the actuator 115, the operable mechanism 144, or the operable mechanism 444) of the actuation device, pulling, pushing, or otherwise moving the actuator from a first position to a second position. The user may actuate the actuator with his or her fingers, for example, while holding a handle (e.g., handle 112) of a medical device (e.g., medical device 100). If a user pulls/pushes the actuator towards the handle, this may cause the inner pull wire to move proximally with respect to the outer sheath because the inner pull wire is coupled to the second fixation mechanism. In embodiments in which the position of the actuator is biased (e.g., via biasing mechanism 450), the force applied by a user may be sufficient to overcome a biasing force of a biasing mechanism.

At step 708, the user may relieve pressure on the actuator causing the actuator to move from the second position to the first position. Alternatively, the user may push or pull the actuator to move it to the first position from the second position. In embodiments of the medical device having a biasing mechanism (such as the biasing mechanism 450), the actuator may pivot back toward the biased position (e.g., the first position). This may cause the inner pull wire to move distally with respect to the outer sheath. In some embodiments, the actuator may not be biased in any position and a user may move the actuator among each of the positions with his or her fingers, puling and pushing the actuator.

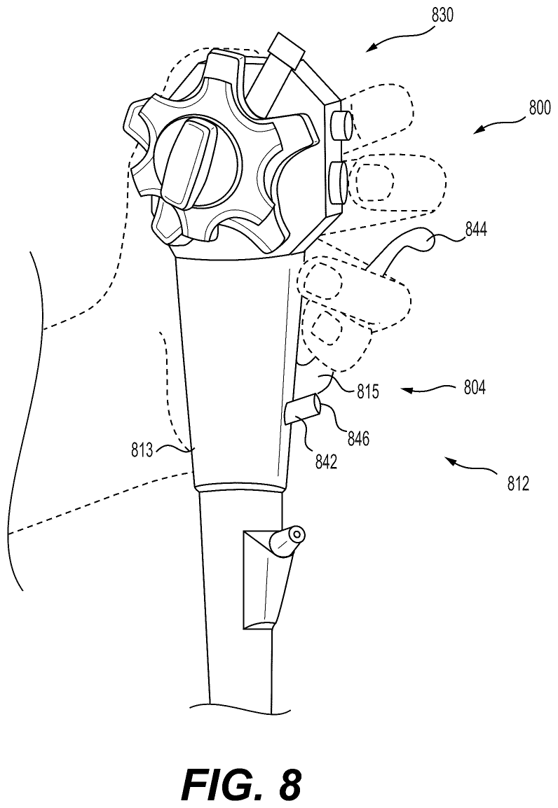

FIG. 8 shows an embodiment of a medical device 800 including a handle 812 and control mechanisms 830. Unless otherwise specified herein, medical device 800 may have any of the operational or structural properties and features of medical device 100. The handle 812 may include an actuation assembly 804, which may have any of the features of the actuation devices 104, 404, described above, unless otherwise specified herein. Whereas the actuation devices 104, 404 may be detachably couplable to handle 112, actuation assembly 804 may be integrally formed with (built directly into) the handle 812. In other words, the actuation assembly 804 may be fixedly, non-removably coupled to the handle 812. In some examples, a material of a housing 813 of the handle 812 may form one or more elements of the actuation assembly 804. In other examples, the actuation assembly 804 may be formed from a separate structure and fixedly, non-removably coupled to the handle 812.

The actuation assembly 804 may include any of the features discussed above. The actuation assembly 804 may include a first fixation mechanism 842, having any of the properties of fixation mechanisms 142, 442, 642, and/or 646. The actuation assembly 804 may further include an actuator 815, having any of the properties of the actuators 115, 415. The actuator 815 may include an operable mechanism 844 (e.g., a lever), having any of the properties of the operable mechanisms 144, 444. The actuator 815 may include a second fixation mechanism 846, having any of the properties of fixation mechanisms 146, 446, 642, and/or 646.

The actuation assembly 804 may function according to any of the principles described above, For example, the first fixation mechanism 842 may couple a first member (e.g., a tube/sheath/coil pipe) of an accessory device (not shown in FIG. 8 but having any of the properties of the devices disclosed above) to the actuation assembly 804. The second fixation mechanism 846 may couple a proximal end/portion of a second member (e.g., an actuation wire or cable) of the accessory device (having any of the properties of the devices discussed above) to the actuation assembly 804. The first member of the accessory device may be fixed relative to the handle 812 via the first fixation mechanism 842, and the second member of the accessory device may be movable relative to the handle 812 via the second fixation mechanism 846. Thus, the actuation assembly 804 may be utilized to actuate the accessory device, as discussed above.

Integrating the actuation assembly 804 into the handle 812 can have advantages, for example, including simplicity of operation, transport, or other advantages. Because the actuation device 804 may be compatible with a wide variety of accessory devices, the actuation device 804 may provide a familiar, same actuation process to an operator for a range of accessory devices. The medical device 800 may also streamline assembly of accessory devices, which may no longer require handles.

It should now be understood that an accessory actuation device can be removably affixed to a handle of a medical device for solo operation of both the medical device and an accessory medical device by a single operator. The accessory actuation device can be biased in a particular position, biasing the accessory device in a given position and enabling better medical procedures. Users of medical devices and medical accessory devices are thus more likely to provide better patient outcomes to their patients because all or more of the user's operational requirements can be performed alone without the requirement for interaction with other medical professionals.

Embodiments of the present disclosure may be applicable to various and different medical or non-medical procedures. In addition, certain aspects of the aforementioned embodiments may be selectively used in collaboration, or removed, during practice, without departing from the scope of the disclosure.

While principles of this disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. An actuation device comprising:
a device body that comprises a first fixation mechanism configured to affix a proximal end of a first member of a medical device to the device body, and
an actuator comprising an operable mechanism and a second fixation mechanism configured to affix a proximal end of a second member of the medical device to the actuator, wherein movement of the operable mechanism from a first position to a second position is configured to move the second member proximally relative to the first member, and wherein movement of the operable mechanism from the second position to the first position is configured to move the second member distally relative to the first member, wherein
at least one of the first member or the second member of the medical device is configured to be removably affixed to the device body or the actuator, respectively.

2. The actuation device of claim 1, wherein:
the first fixation mechanism comprises a first arm and a second arm, and
the first member is configured to extend between the first arm and the second arm.

3. The actuation device of claim 1, wherein the actuator is pivotally mounted relative to the device body, and wherein the actuator comprises a lever that can move the actuator around a pivot.

4. The actuation device of claim 3, wherein a portion of the second fixation mechanism extends approximately perpendicularly to a portion of the lever.

5. The actuation device of claim 1, wherein the device body is removably fixable to an endoscope handle.

6. The actuation device of claim 1, further comprising a biasing member configured to bias the actuator in the first position or the second position.

7. The actuation device of claim 6, wherein the biasing member is a torsion spring.

8. The actuation device of claim 1, wherein the second fixation mechanism comprises a pair of arms, each arm comprising a curved portion.

9. The actuation device of claim 1, wherein the actuation device is configured to removably affix to an endoscope handle and the endoscope handle comprises a female mating member and the device body comprises a corresponding male mating member for removably coupling the device body to the endoscope handle.

10. The actuation device of claim 9, wherein the male mating member is formed of a flexible material.

11. The actuation device of claim 1, wherein the medical device is a first medical device, and wherein the actuation device is integrally formed with a handle of a second medical device.

12. The actuation device of claim 1, wherein the medical device is a first medical device, and wherein the actuation device is removably fixable to a handle of a second medical device.

13. The actuation device of claim 1, wherein the first member is a sheath and the second member is a pull wire.

14. The actuation device of claim 1, wherein the second member includes at least one protrusion, and wherein the second fixation mechanism includes at least one hook.

15. The actuation device of claim 1, wherein the medical device is a first medical device, wherein the first fixation mechanism is configured to fix the first member of the first medical device relative to a handle of a second medical device, and wherein the second fixation mechanism is movable with respect to the first fixation mechanism, such that the second member of the first medical device is movable with respect to the first member of the first medical device and the handle of the second medical device.

16. A medical system comprising:
a first medical device having a first handle;
an actuation device comprising:
a device body that comprises a first fixation mechanism configured to affix a proximal end of a first member of a second medical device to the device body, and
an actuator comprising an operable mechanism and a second fixation mechanism configured to affix a proximal end of a second member of the second medical device to the actuator, wherein movement of the operable mechanism from a first position to a second position is configured to move the second member relative to the first member, and wherein movement of the operable mechanism from the second position to the first position is configured to move the second member relative to the first member, wherein
the actuation device is operable from the handle of the first medical device, and
at least one of the first member or the second member of the second medical device is configured to be removably affixed to the device body or the actuator, respectively.

17. The medical system of claim 16, further comprising a third medical device, wherein a first member of the third medical device is removably affixable to the first fixation member, and wherein a second member of the third medical device is affixable to the second fixation member.

18. The medical system of claim 17, wherein the second medical device is one of a tome, a snare, a balloon, a basket, a knife, a needle, a ligation device, a clip, forceps, graspers, a stapler, a suturing device, a stent, a cautery device, or a biopsy collection device, and the third medical device is another of a tome, a snare, a balloon, a basket, a knife, a needle, a ligation device, a clip, forceps, graspers, a stapler, a suturing device, a stent, a cautery device, or a biopsy collection device, different from the second medical device.

19. An actuation device comprising:
a device body that comprises a first fixation mechanism configured to removably affix a first member of a medical device to the device body, and
a lever fixedly coupled to a second fixation mechanism, wherein the second fixation mechanism is configured to removably affix a proximal end of a second member of the medical device to the lever, wherein movement of the lever from a first position to a second position is configured to move the second member proximally relative to the first member, and wherein movement of the lever from the second position to the first position is configured to move the second member distally relative to the first member.

20. The actuation device of claim 19, wherein the first fixation mechanism includes at least one prong, wherein the first member includes at least one protrusion, wherein the second fixation mechanism includes at least one hook, and wherein the second member includes at least one protrusion.
